(19) United States
(12) Patent Application Publication
(10) Pub. No.: US 2024/0074959 A1
(43) Pub. Date: Mar. 7, 2024

(54) WATER-IN-OIL TYPE COMPOSITION

(71) Applicant: SHISEIDO COMPANY, LTD., Tokyo (JP)

(72) Inventors: Keita NISHIDA, Tokyo (JP); Saki KAMADA, Tokyo (JP); Haruka NISHI, Tokyo (JP); Chije PARK, Tokyo (JP)

(21) Appl. No.: 18/261,512

(22) PCT Filed: Feb. 7, 2022
(86) PCT No.: PCT/JP2022/004647

§ 371 Date: Jul. 14, 2023

(30) Foreign Application Priority Data
Feb. 16, 2021 (JP) 2021-022875

(51) Int. Cl. A61K 8/37; A61K 8/06; A61K 8/25; A61K 8/31; A61K 8/92; A61Q 1/00; A61Q 17/04

(52) U.S. Cl. CPC A61K 8/37; A61K 8/064; A61K 8/25; A61K 8/31; A61K 8/922; A61Q 1/00; A61Q 17/04

(57) ABSTRACT

A water-in-oil type composition, including (A) an organic modified clay mineral, (B) 3% by mass or greater of a polar oil having IOB of 0.3 or higher, (C) a nonpolar oil having an alkyl side chain, (D1) a polyoxyethylene hardened castor oil, and (D2) an emulsification activating agent, that is a compound having an alkyl side chain containing 10 or more carbon atoms, or a compound having a fatty acid side chain containing 10 or more carbon atoms or a polyoxyethylene adduct of the compound.

### TECHNICAL FIELD

The present invention relates to a water-in-oil type composition.

### BACKGROUND OF THE INVENTION

Water-in-oil type compositions in which an oil-based component constitutes the external phase and a water-based component constitutes the internal phase are widely used as, for example, cosmetic agents and quasi-drugs because they have, for example, excellent moisture-retaining and occlusive properties when applied on skin. In order for the water-in-oil type compositions to exert their functions appropriately, it is important that an emulsified state of the oil-based component and the water-based component is stable. Hence, various ingenious ideas for stabilization are being explored.

For example, use of an organic modified clay mineral as an emulsifier is known, as described in Patent Document 1. When used in combination with a surfactant appropriately, an organic modified clay mineral can bring a composition into a stable gel state.

### RELATED-ART DOCUMENT

### Patent Document

Patent Document 1: Japanese Patent Application Laid-Open NO. 2013-107865

### SUMMARY OF THE INVENTION

### Problems to be Solved by the Invention

Water-in-oil type compositions are often used as skin application agents having an ultraviolet protection function (e.g., cosmetic agents and personal care products). In this case, prescriptions of the compositions include a polar oil having a high ultraviolet absorbing effect. However, when the content of a polar oil is increased, it becomes difficult to stably retain the emulsified state of the oil-based component and the water-based component, and the gel state obtained by use of an organic modified clay mineral.

In view of the above point, an object of an embodiment of the present invention is to improve stability of the state of a water-in-oil type composition containing a polar oil.

### Means for Solving the Problems

An embodiment of the present invention for solving the problem described above is a water-in-oil type composition containing (A) an organic modified clay mineral, (B) 3% by mass or greater of a polar oil having IOB of 0.3 or higher, (C) a nonpolar oil having an alkyl side chain, (D1) a polyoxyethylene hardened castor oil, and (D2) an emulsification activating agent, that is a compound having an alkyl side chain containing 10 or more carbon atoms, or a compound having a fatty acid side chain containing 10 or more carbon atoms or a polyoxyethylene adduct of the compound.

### Effects of the Invention

According to an embodiment of the present invention, it is possible to improve stability of the state of a water-in-oil type composition containing a polar oil.

### DETAILED DESCRIPTION OF THE INVENTION

Embodiments of the present invention will be described in detail below. However, the present invention is not limited to the embodiments described below.

### <Water-In-Oil Type Composition>

A water-in-oil type composition according to a first embodiment of the present invention contains (A) an organic modified clay mineral, (B) 3% by mass or greater of a polar oil having IOB of 0.3 or higher, (C) a nonpolar oil having an alkyl side chain, (D1) a polyoxyethylene hardened castor oil, and (D2) an emulsification activating agent, that is a compound having an alkyl side chain containing 10 or more carbon atoms, or a compound having a fatty acid side chain containing 10 or more carbon atoms or a polyoxyethylene adduct of the compound.

A water-in-oil type composition according to a second embodiment of the present invention contains (A) an organic modified clay mineral, (B) 3% by mass or greater of a polar oil having IOB of 0.3 or higher, (C) a nonpolar oil having an alkyl side chain, and (D) a fatty acid ester of a polyoxyethylene hardened castor oil.

The water-in-oil type compositions according to the embodiments of the present invention are solid or semisolid, and may be provided in a state referred to as a gel state or a jelly state, a cream state, or a balm state. A water-in-oil type composition having such a state has a high skin protection or occlusive effect because it can be easily applied as a skin application agent and can form a coating membrane having a certain degree of thickness on the surface of skin.

### First Embodiment

### <(A) Organic Modified Clay Mineral>

(A) The organic modified clay mineral is not particularly limited so long as it is commonly used in skin application agents. (A) The organic modified clay mineral may be, for example, a cation-modified clay mineral obtained by treating a layered clay mineral such as bentonite, laponite, hectorite, montmorillonite, and magnesium aluminum silicate with a quaternary ammonium salt-type cationic surfactant. Specific examples of (A) the organic modified clay mineral include dimethyl distearyl ammonium hectorite (distearyl dimonium hectorite), dimethyl alkyl ammonium hectorite, benzyl dimethyl stearyl ammonium hectorite, and distearyl dimethyl ammonium chloride-treated magnesium aluminum silicate. Examples of commercially available products include BENTONE 27 (benzyl dimethyl stearyl ammonium chloride-treated hectorite: available from National Lead Co., and Rheox, Inc.), BENTONE 34 (chemical name: quaternium-18 bentonite: available from Rheox, Inc.), BENTONE 38 (distearyl dimethyl ammonium chloride-treated hectorite: available from National Lead Co.), BENTONE 38V (quaternium-18 hectorite: available from Rheox, Inc.), CLAYTONE 40 (available from Southern Clay Products Inc.), and CLAYTONE SO (available from Southern Clay Products Inc.). Among those above, it is preferable to use dimethyl distearyl ammonium hectorite and benzyl dimethyl stearyl ammonium hectorite, and it is more preferable to use dimethyl distearyl ammonium hectorite. One of these (A) organic modified clay minerals may be used alone or two or more of them may be used in combination.

(A) The organic modified clay mineral can function as an emulsifier that assists emulsification of an oil-based component and a water-based component. More specifically, addition of (A) the organic modified clay mineral enables favorable thickening or gelation of an oil-based component, and retention of an oil phase in a gelated state over time. This in turn makes a dispersed state of a water phase stable, and enables stable retention of the entire water-in-oil type composition in a solid or semisolid state, or in a gelated state for a long term.

The content of (A) the organic modified clay mineral is preferably 0.05% by mass or greater and 10% by mass or less, and more preferably 0.1% by mass or greater and 5% by mass or less relative to the whole amount of the water-in-oil type composition. When the content of (A) the organic modified clay mineral is 0.05% by mass or greater, a sufficient stability of state can be imparted to the water-in-oil type composition. When the content of (A) the organic modified clay mineral is 10% by mass or less, a good touch and feel with little stickiness is obtained during use, and convenience of use, such as smooth spread over skin, is also improved.

### <(B) Polar Oil>

(B) The polar oil may be an oil-based component commonly used in skin application agents and having a relatively high polarity. The IOB value of (B) the polar oil used in the present embodiment may be 0.3 or higher. The IOB value of (B) the polar oil may be preferably 0.8 or lower, and more preferably 0.7 or lower. When (B) the polar oil contains an ultraviolet absorbing agent (described below), the IOB value of the ultraviolet absorbing agent is preferably 0.3 or higher and 0.7 or lower. When the IOB value of (B) the polar oil is 0.3 or higher, the gel state of the water-in-oil type composition can be stabilized, and the stabilized state can be retained over time.

The IOB value is the abbreviation of Inorganic/Organic Balance, which is a value indicating the ratio of an inorganic value to an organic value and serves as an indicator of the degree of polarity of an organic compound. Specifically, the IOB value is represented by a formula “IOB value=inorganic value/organic value”. An “inorganic value” and an “organic value” are defined depending on each atom or functional group in a molecule, such as an “organic value” of 20 for one carbon atom, and an “inorganic value” of 100 for one hydroxyl group. It is possible to calculate the IOB value of an organic compound, by adding up the “inorganic values” and the “organic values” of all atoms and functional groups in the compound (e.g., see Fujita, “Kagaku no ryouiki” (Provisional translation: Area of Chemistry), 11 (10), pp. 719-725, 1957). There is a tendency that the higher the IOB value, the higher the inorganic property is and the higher the hydrophilicity is. When (B) the polar oil is a combination of two or more having different IOB values, the IOB value of (B) the polar oil is a weighted average.

The content of (B) the polar oil may be 3% by mass or greater relative to the whole amount of the water-in-oil type composition. The content of (B) the polar oil may be preferably 5% by mass or greater, and more preferably 7% by mass or greater relative to the whole amount of the composition, depending on the function or use of the composition. When the lower limit of the content of (B) the polar oil is the value specified above, for example, a moisture-retaining effect or an occlusive effect (occlusion effect) can be enhanced. Moreover, according to the composition of the present embodiment, it is possible to stabilize the water-in-oil type emulsified state of the composition even when the composition contains a relatively high content of (B) the polar oil as in the range specified above.

The content of (B) the polar oil may preferably be 30% by mass or less, or more preferably 20% by mass or less relative to the whole amount of the composition. When the upper limit of the content of (B) the polar oil is the value specified above, it is possible to improve the stability of the dosage form, and to inhibit stickiness during application and provide a better touch and feel during use.

It is preferable that (B) the polar oil contains an ultraviolet absorbing agent, i.e., a polar oil having an ultraviolet absorbing function. When (B) the polar oil contains an ultraviolet absorbing agent, the water-in-oil type composition according to the present embodiment can be suitably used as a cosmetic composition having a sunscreen effect, or a composition for sunscreen.

The type of the ultraviolet absorbing agent contained in (B) the polar oil is not particularly limited, and may be, for example, benzoic acid derivatives, salicylic acid derivatives, cinnamic acid derivatives, dibenzoyl methane derivatives, β,β-diphenyl acrylate derivatives, benzophenone derivatives, benzylidene camphor derivatives, phenyl benzoimidazole derivatives, triazine derivatives, phenyl benzotriazole derivatives, anthranil derivatives, imidazoline derivatives, benzal malonate derivatives, and 4,4-diaryl butadiene derivatives. Specific examples of the ultraviolet absorbing agent include octyl salicylate (IOB=0.6), octocrylene (IOB=0.32), homosalate (IOB=0.6), and ethyl hexyl methoxycinnamate (IOB=0.35), and octyl salicylate (IOB=0.6), octocrylene (IOB=0.32), and homosalate (IOB=0.6) are preferable. One of these ultraviolet absorbing agents may be used alone, or two or more of these ultraviolet absorbing agents may be used in combination.

The content of the ultraviolet absorbing agent relative to the whole amount of (B) the polar oil may be 100% by mass, i.e., (B) the polar oil in the water-in-oil type composition according to the present embodiment may be the ultraviolet absorbing agent.

The content of the ultraviolet absorbing agent is preferably 3% by mass or greater, more preferably 5% by mass or greater, and yet more preferably 7% by mass or greater relative to the whole amount of the water-in-oil type composition. The content of an ultraviolet-absorbing polar oil may be preferably 20% by mass or less relative to the whole amount of the water-in-oil type composition. When the content of the ultraviolet absorbing agent is in the range specified above, it is possible to provide a water-in-oil type composition that has a stable state while also having an ultraviolet protection function.

Specific examples of ester oils include diisopropyl sebacate (IOB=0.4), glyceryl tri-2-ethylhexanoate (triethylhexanoin) (IOB=0.35), tripropylene glycol dipivalate (IOB=0.52), pentaerythritol tetra-2-ethylhexanoate (IOB=0.35), diethylhexyl succinate (IOB=0.32), neopentyl glycol diethylhexanoate (IOB=0.32), trimethylolpropane triethylhexanoate (IOB=0.33), pentaerythrityl tetraethylhexanoate (IOB=0.35), diisopropyl adipate (IOB=0.46), diisostearyl malate (IOB=0.28), tripropylene glycol dineopentanoate (IOB=0.52), propylene glycol dicaproate (IOB=0.32), and glyceryl triethylhexanoate (IOB=0.36). Among these ester oils, diisopropyl sebacate (IOB=0.4), glyceryl tri-2-ethylhexanoate (triethylhexanoin) (IOB=0.35), tripropylene glycol dipivalate (IOB=0.52), pentaerythritol tetra-2-ethylhexanoate (IOB=0.35), and diethylhexyl succinate (IOB=0.32) are preferable. One of these ester oils may be used alone or two or more of these ester oils may be used in combination.

When the water-in-oil type composition contains an ester oil, the emulsification balance between an oil-based component and a water-based component can be adjusted, and dissolution of the ultraviolet absorbing agent can be promoted.

As clear from the specific examples of (B) the polar oil given above, (B) the polar oil may be solid or liquid at normal temperature.

### <(C) Nonpolar Oil>

(C) The nonpolar oil represents an oil-based component having a relatively low polarity, and may be an oil-based component having IOB of lower than 0.3. Examples of (C) the nonpolar oil include hydrocarbon oils, nonpolar silicone oils, and nonpolar ester oils. As the nonpolar oil in the present embodiment, one that has a structure having an alkyl side chain is preferable. Here, a “side chain” represents a chain portion directly bound to a main chain, which is the longest straight line-shaped portion of a chain molecule. By blending (C) the nonpolar oil having an alkyl side chain, it is possible to stabilize a gel state of the water-in-oil type composition for a long term.

(C) The nonpolar oil may be a monomer or a polymer (including an oligomer). Whichever of a monomer and a polymer (C) the nonpolar oil is, the number of carbon atoms in one alkyl side chain may be 1, preferably 2 or greater, more preferably 3 or greater, and yet more preferably 5 or greater. An alkyl group in the alkyl side chain may be a straight-chained or branched alkyl group.

When (C) the nonpolar oil is a monomer, the number of alkyl side chains bound to the nonpolar oil per molecule may be 1, preferably 2 or greater, more preferably 3 or greater, and yet more preferably 5 or greater. When (C) the nonpolar oil is a polymer, the number of alkyl side chains in one monomer unit may be 1, or 2 or greater.

When (C) the nonpolar oil is a monomer, the total number of carbon atoms contained in alkyl side chains of one molecule (i.e., the total number of carbon atoms contained in all alkyl side chains, when there are two or more alkyl side chains) may be 1, preferably 2 or greater, and more preferably 3 or greater, and also preferably 10 or less, and more preferably 8 or less. When (C) the nonpolar oil is a polymer, the total number of carbon atoms contained in alkyl side chains of one monomer unit may be 1, preferably 2 or greater, more preferably 3 or greater, and yet more preferably 5 or greater, and also preferably 20 or less, and more preferably 10 or less.

When (C) the nonpolar oil is a hydrocarbon oil having an alkyl side chain, it may be an aliphatic unsaturated hydrocarbon oil that is liquid or solid at normal temperature.

Specific examples of the hydrocarbon oil having an alkyl side chain include hydrogenated polydecene, hydrogenated polyisobutene, squalane, isohexadecane, and isododecane. Among these hydrocarbon oils, hardened polydecene is preferable. One of these hydrocarbon oils may be used alone or two or more of these hydrocarbon oils may be used in combination.

Examples of nonpolar silicones having an alkyl side chain include chain polysiloxane, e.g., silicone compounds obtained by introducing an alkyl side chain into dimethyl polysiloxane. Any other functional group than alkyl may be introduced. However, it is preferable that alkyl is the only functional group introduced as a side chain. Silicones free of an aromatic functional group are preferable.

A specific example of the nonpolar silicone having an alkyl side chain is caprylyl methicone. One of these silicone oils may be used alone or two or more of these silicone oils may be used in combination.

When (C) the nonpolar oil is a nonpolar ester oil, a specific example of the nonpolar ester oil is cetyl 2-ethylhexanoate.

The content of (C) the nonpolar oil is preferably 5% by mass or greater and 40% by mass or less, and more preferably 10% by mass or greater and 30% by mass or less relative to the whole amount of the water-in-oil type composition. When the content of (C) the nonpolar oil is in the range specified above, it is possible to stabilize a gel state of the water-in-oil type composition for a long term, and to improve touch and feel during application.

The ratio of the content of (C) the nonpolar oil to the content of (B) the polar oil ([content of (C) nonpolar oil]/[content of (B) polar oil)]) is preferably 0.25 or greater and 4 or less, more preferably 0.3 or greater and 3.5 or less, and yet more preferably 0.4 or greater and 3.0 or less. When the ratio of the content of (C) the nonpolar oil to the content of (B) the polar oil is in the range specified above, the balance between (C) the nonpolar oil and (B) the polar oil is improved, making it possible to gelate an oil phase stably, and to retain a gelated state of the water-in-oil type composition favorably.

### <(D1) Polyoxyethylene Hardened Castor Oil>

(D1) The polyoxyethylene (POE) hardened castor oil (polyoxyethylene hydrogenated castor oil) is an adduct of a castor oil with hydrogen and polyethylene glycol (PEG). (D1) The polyoxyethylene hardened castor oil can function as a nonionic surfactant, and has a high performance in dispersing mainly (A) the organic modified clay mineral in the oil phase of the water-in-oil type composition according to the present embodiment.

The HLB value of (D1) the polyoxyethylene hardened castor oil used in the present embodiment is not particularly limited. However, in order to retain a water-in-oil type emulsified state favorably, the HLB value may be preferably 12 or lower. The “HLB value” means a Hydrophilic-Lipophilic Balance (HLB) value indicating the degree of affinity of a surfactant with water and oils, and may be calculated according to Griffin's formula (HLB value=molecular weight of glycerin moiety×20/whole molecular weight).

The number of moles of polyoxyethylene added to (D1) the polyoxyethylene hardened castor oil is preferably 1 or greater and 60 or less, and more preferably 5 or greater and 40 or less.

Specific examples of (D1) the polyoxyethylene hardened castor oil include a POE (5) hardened castor oil, a POE (10) hardened castor oil, a POE (20) hardened castor oil, a POE (30) hardened castor oil, a POE (40) hardened castor oil, and a POE (60) hardened castor oil. Among these polyoxyethylene hardened castor oils, a POE (10) hardened castor oil is preferable.

One of (D1) the polyoxyethylene hardened castor oils mentioned above may be used alone, or two or more of them may be used in combination.

The content of (D1) the polyoxyethylene hardened castor oil is preferably 0.05% by mass or greater and 10% by mass or less, and more preferably 0.1% by mass or greater and 5% by mass or less relative to the whole amount of the water-in-oil type composition. When the content of (D1) the polyoxyethylene hardened castor oil is in the range specified above, the gelation effect or the thickening effect by (A) the organic modified clay mineral is promoted, and a gel state of the water-in-oil type composition can be retained stably.

The ratio of the content of (D1) the polyoxyethylene hardened castor oil to the content of (A) the organic modified clay mineral ([content of (D1) polyoxyethylene hardened castor oil]/[content of (A) organic modified clay mineral]) is preferably 0.1 or greater and 5 or less, and more preferably 0.5 or greater and 2 or less. When the ratio of the content of (D1) the polyoxyethylene hardened castor oil to the content of (A) the organic modified clay mineral is in the range specified above, gelation of the oil-based component is promoted, facilitating retention of also the state of the entire water-in-oil type composition in a gel state.

### <(D2) Emulsification Activating Agent>

The water-in-oil type composition according to the present embodiment further contains, as (D2) the emulsification activating agent, an emulsification activating agent, that is a compound having an alkyl side chain containing 10 or more carbon atoms, or a compound having a fatty acid side chain containing 10 or more carbon atoms or a polyoxyethylene adduct of the compound. (D2) The emulsification activating agent mainly promotes emulsification of an oil-based component and a water-based component, and has a high performance in retaining an emulsified state over time.

More specifically, (D2) the emulsification activating agent may be a silicone-based emulsification activating agent having an alkyl side chain containing or more carbon atoms. An alkyl group in the alkyl side chain may be a straight-chained or branched alkyl group. In addition to the alkyl side chain, the silicone-based emulsification activating agent may also have a polyether side chain (i.e., a functional group side chain derived from polyethylene glycol or polypropylene glycol), and may further have a silicone side chain.

When (D2) the emulsification activating agent is a silicone-based emulsification activating agent, the number of carbon atoms in one alkyl side chain is preferably 10 or greater and 20 or less, and more preferably 12 or greater and 15 or less. An alkyl group in the alkyl side chain may be a straight-chained or branched alkyl group.

Specific examples of the silicone-based emulsification activating agent include cetyl PEG/PPG-10/1 dimethicone, and lauryl PEG-9 polydimethyl siloxyethyl dimethicone.

(D2) The emulsification activating agent may be a fatty acid ester of glycerin or polyglycerin having a fatty acid side chain containing 10 or more carbon atoms, or a polyoxyethylene adduct of the fatty acid ester. In the present specification, a fatty acid side chain is a functional group derived from a fatty acid or a fatty acid condensate, and, more specifically, may be a functional group obtained by removing a hydrogen atom from a carboxyl group of a fatty acid, or a functional group obtained by removing a hydrogen atom from a carboxyl group at an end of a condensate of an unsaturated fatty acid or a hydroxy fatty acid.

When the fatty acid side chain is derived from a fatty acid monomer, the number of carbon atoms in the fatty acid is preferably 10 or greater and 24 or less, more preferably 14 or greater and 22 or less, and yet more preferably 16 or greater and 20 or less. Also when the fatty acid side chain is derived from a fatty acid condensate, the number of carbon atoms in the fatty acid (i.e., the fatty acid before condensation) may be 10 or greater and 24 or less, more preferably 14 or greater and 22 or less, and yet more preferably 16 or greater and 20 or less.

When (D2) the emulsification activating agent is a polyoxyethylene adduct, the number of moles of polyoxyethylene is preferably 5 or greater and 60 or less, and more preferably 10 or greater and 40 or less.

When (D2) the emulsification activating agent is a polyoxyethylene adduct, polyoxyethylene may be added to the fatty acid side chain itself, but the polyoxyethylene adduct may be free of a polyethylene glycol chain branched from the fatty acid side chain. Alternatively, the fatty acid constituting the fatty acid side chain may be an unsubstituted fatty acid, more specifically, a fatty acid free of a hydroxyl group.

When (D2) the emulsification activating agent is a fatty acid ester of glycerin or polyglycerin having a fatty acid side chain containing 10 or more carbon atoms, or a polyoxyethylene adduct of the fatty acid ester, examples of (D2) the emulsification activating agent include: polyoxyethylene adducts of fatty acid triglyceride, such as POE (10) glycerin triisostearate and POE (15) glycerin triisostearate; fatty acid esters of polyglycerin, such as polyglyceryl-10 pentaoleate, polyglyceryl-2 triisostearate, and polyglyceryl-10 pentastearate; and esters of hydroxy fatty acid condensates with polyglycerin, such as polyglyceryl-6 polyricinoleate and polyglyceryl-6 polyhydroxystearate.

The HLB value of (D2) the emulsification activating agent is preferably 9 or lower, more preferably 7.5 or lower, and yet more preferably 6 or lower.

Whichever of the compound having an alkyl side chain containing 10 or more carbon atoms, and the compound having a fatty acid side chain containing 10 or more carbon atoms or a polyoxyethylene adduct of the compound (D2) the emulsification activating agent is, the content of (D2) the emulsification activating agent is preferably 0.05% by mass or greater and 10% by mass or less, and more preferably 0.1% by mass or greater and 5% by mass or less relative to the whole amount of the water-in-oil type composition. When (D2) the emulsification activating agent is contained in the range specified above, it is possible to retain a good emulsified state of an oil-based component and a water-based component for a long term.

### <Other Components>

In addition to the components (A) to (D2) described above, the water-in-oil type composition according to the present embodiment contains water commonly used in compositions for external application. For example, purified water, ion-exchanged water, and tap water may be used as the water. The content of the water in the present embodiment may be preferably 50% by mass or greater and 80% by mass or less and more preferably 55% by mass or greater and 75% by mass or less relative to the whole amount of the water-in-oil type composition.

The water-in-oil type composition may further contain desirably selected components other than the components (A) to (D2) and the water described above, to an extent that the effects of the present embodiment are not inhibited. For example, the water-in-oil type composition may contain, for example, a water-soluble alcohol as a water-based component, and may contain, for example, a higher alcohol, a liquid fat or oil, a solid fat or oil, a wax, a higher fatty acid, or a flagrance as an oil-based component

The water-in-oil type composition according to the present embodiment may contain a wax. A wax is an oil-based component that is solid or semisolid at normal temperature, contains a hydrocarbon, a neutral fat, a higher fatty acid, and an ester of a higher fatty acid and a higher alcohol, and may function to increase the stability of the dosage form of the composition. From a viewpoint that the water-in-oil type composition to be obtained would have a light touch and feel during use, it is preferable that the content of the wax is low, and may be preferably 1% by mass or less, more preferably less than 0.5% by mass, and yet more preferably 0.1% by mass or less relative to the whole amount of the water-in-oil type composition. Further, in interest of saving time on heating during production, it is preferable that the content of the wax is 0% by mass, i.e., that the water-in-oil type composition is free of a wax. To put it another way, it is possible to obtain a stable water-in-oil type composition without blending a wax, because the water-in-oil type composition according to the present embodiment can form a stable gel based on the blend of the components (A), (B), (C), (D1), and (D2) described above.

The water-in-oil type composition according to the present embodiment may contain a cyclic silicone or a cyclic polysiloxane (e.g., octamethylcyclotetrasiloxane, decamethylcyclopentasiloxane, and dodecamethylcyclohexasiloxane). However, when the water-in-oil type composition contains a cyclic silicone, the content of the cyclic silicone may need only be 1% by mass or less, more preferably 0.5% by mass or less, yet more preferably 0.1% by mass or less, and still more preferably 0.01% by mass or less relative to the whole amount of the water-in-oil type composition. It is preferable that the water-in-oil type composition substantially does not contain or does not contain a cyclic silicone. In the present specification, a predetermined component “substantially being not contained” means that an embodiment containing the predetermined component that is unavoidably mixed during extraction of raw materials or a production step of the composition is encompassed.

The water-in-oil type composition may also contain a surfactant such as a cationic surfactant, an anionic surfactant, any other nonionic surfactant than those described above, and an amphoteric surfactant. The water-in-oil type composition may also contain a thickener, a moisture-retaining agent, a percutaneous absorption inhibitor, a chelate agent, a pigment, an antioxidant, an antiseptic agent, an anti-inflammatory agent, a whitening agent, a plant extract, an activator agent, a blood circulation accelerator, and an antiseborrheic agent.

Moreover, the water-in-oil type composition according to the present embodiment may contain an organic powder or an inorganic powder. For example, specific examples of inorganic powders include titanium oxide, zinc oxide, cerium oxide, talc, silica, mica, sericite, kaolin, titanium mica, black iron oxide, yellow iron oxide, colcothar, ultramarine blue, Prussian blue, chromium oxide, and chromium hydroxide. A complex powder obtained by coating particles of any other than titanium oxide with titanium oxide may also be contained. Moreover, a powder (having an average particle diameter of 1 μm or greater and 20 μm or less) of a biodegradable resin such as polyhydroxyalkanoate, such as polyhydroxybutyric acid, and poly(3-hydroxybutyric acid-co-3-hydroxyvaleric acid) may also be contained.

### <Use>

The use of the water-in-oil type composition according to the present embodiment is not particularly limited, and the water-in-oil type composition can be suitably used as an agent for external application or an agent for skin application, particularly as a cosmetic agent in the fields of cosmetic agents, quasi-drugs, and personal care products. Moreover, the water-in-oil type composition according to the present embodiment may be, for example, a sunscreen for ultraviolet protection, or a cosmetic agent having an increased ultraviolet protection function, such as a basic skin-care item such as a foundation or a makeup item.

### Second Embodiment

A composition according to a second embodiment contains (A) an organic modified clay mineral, (B) 3% by mass or greater of a polar oil having IOB of 0.3 or higher, and (C) a nonpolar oil having an alkyl side chain, which are the same as those in the first embodiment, but contains (D) a fatty acid ester of a polyoxyethylene hardened castor oil instead of (D1) the polyoxyethylene hardened castor oil and (D2) the emulsification activating agent of the first embodiment. When (D) the fatty acid ester of the polyoxyethylene hardened castor oil is contained, uniform dispersion of (A) the organic modified clay mineral in an oil-based component is promoted, and the emulsification balance between the oil-based component and a water-based component of the water-in-oil type composition can be retained favorably. Hence, it is possible to stabilize the state of the water-in-oil type composition for a long term.

The fatty acid that constitutes (D) the fatty acid ester of the polyoxyethylene hardened castor oil may be a higher fatty acid, and the number of carbon atoms in the higher fatty acid may be preferably 13 or greater and 25 or less, and more preferably 16 or greater and 20 or less. The fatty acid may have a straight-chained or branched structure. However, a branched fatty acid is preferable, and isostearic acid is particularly preferable.

The number of moles of polyoxyethylene added to (D) the fatty acid ester of the polyoxyethylene hardened castor oil is preferably 5 or greater and 60 or less, and more preferably 10 or greater and 40 or less.

Specific examples of (D) the fatty acid ester of the polyoxyethylene hardened castor oil includes polyoxyethylene adducts of triisostearic acid, such as a triisostearic acid PEG-10 hardened castor oil and a triisostearic acid PEG-20 hardened castor oil.

The content of (D) the fatty acid ester of the polyoxyethylene hardened castor oil may be preferably 0.1% by mass or greater and 15% by mass or less, and more preferably 1% by mass or greater and 10% by mass or less relative to the whole amount of the water-in-oil type composition. When the content is in the range specified above, an emulsified state of an oil-based component and a water-based component can be retained favorably for a long term.

The ratio of the content of (D) the fatty acid ester of the polyoxyethylene hardened castor oil to the content of (A) the organic modified clay mineral ([content of (D) polyoxyethylene hardened castor oil]/[content of (A) organic modified clay mineral]) may be preferably 0.2 or greater and 10 or less, and more preferably 1 or greater and 5 or less. When the ratio of the content of (D) the polyoxyethylene hardened castor oil to the content of (A) the organic modified clay mineral is in the range specified above, gelation of an oil-based component is promoted, an emulsified state of the oil-based component and a water-based component is improved, and retention of also the state of the entire water-in-oil type composition in a gel state is facilitated.

Other components that can be blended in the water-in-oil type composition according to the second embodiment and the use of the composition are the same as those described in the first embodiment.

### EXAMPLES

Compositions having the compositions presented in Table 1 and Table 2 were prepared by a routine method. That is, a polar oil, a nonpolar oil, a polyoxyethylene hardened castor oil, and an emulsification activating agent were mixed, an organic modified clay mineral was next added to the resulting product and the resulting product was further mixed, and then water was added to the resulting product, to obtain compositions of examples 1 to 11 and Comparative Examples 1 to 5. Likewise, a polar oil, a nonpolar oil, and a fatty acid ester of a polyoxyethylene hardened castor oil were mixed, an organic modified clay mineral was next added to the resulting product and the resulting product was further mixed, and water was added to the resulting product, to obtain a composition of Example 12. A gelated state of each of Examples 1 to 12 and Comparative Example 1 to 5 was evaluated. Table 1 and Table 2 also present the evaluation results.

### <Evaluation of Gelation Stability>

Gelation stability of the composition of each Example was evaluated. The evaluation was performed by visual observation according to the evaluation criteria described below.
A: It was confirmed that the composition became a good gel state, and stability of the gel state was retained after 28 days passed.
B: It was confirmed that the composition became a good gel state, and stability of the gel state was retained after 7 days passed.
C: It was confirmed that the composition became a good gel state, and stability of the gel state was retained after 1 day passed.
D: The composition did not become a gel state.

TABLE 1

| | | Ex. | Ex. | Ex. | Ex. | Comp. | Ex. | Ex. | Comp. |
|---|---|---|---|---|---|---|---|---|---|
| | Component | 1 | 2 | 3 | 4 | Ex. 1 | 5 | 6 | Ex. 2 |
| (C) | Isododecane | 20 | — | — | — | — | — | — | — |
| Nonpolar | Hydrogenated polydecene (silkflo362) | — | 20 | 20 | 20 | 20 | — | — | — |
| oil | Caprylyl methicone | — | — | — | — | — | 20 | — | — |
| | Squalane | — | — | — | — | — | — | 20 | — |
| | Dimethicone (silicone KF-96L-1.5 cs) | — | — | — | — | — | — | — | 20 |
| (B) | Ethylhexyl salicylate (octyl | 8 | 8 | — | — | — | 8 | 8 | 8 |
| Polar oil | salicylate) (IOB = 0.6) | | | | | | | | |
| | Octocrylene (IOB = 0.32) | — | — | 8 | — | — | — | — | — |
| | Diisopropyl sebacate (IOB = 0.4) | — | — | — | 8 | — | — | — | — |
| | (Eceran 200) | | | | | | | | |
| | Isopropyl myristate (IOB = 0.18) (IFM-R) | — | — | — | — | 8 | — | — | — |
| (D1) | PEG-10 hardened castor oil | — | 2 | 2 | 2 | 2 | 2 | 2 | 2 |
| POE | (Nikkol HC-10) | | | | | | | | |
| hardened | PEG-40 hardened castor oil | 2 | — | — | — | — | — | — | — |
| castor oil | (Nikkol HC-40) | | | | | | | | |
| (D2) | PEG-10 dimethicone | — | — | — | — | — | — | — | — |
| Activating | Lauryl PEG-9 polydimethyl | 2 | 2 | 2 | 2 | 2 | 2 | 2 | 2 |
| agent | siloxyethyl dimethicone (KF-6038) | | | | | | | | |
| | Polyglyceryl-6 polyricinoleate | — | — | — | — | — | — | — | — |
| | Cetyl PEG/PPG-10/1 dimethicone | — | — | — | — | — | — | — | — |
| | (ABIL EM 180) | | | | | | | | |
| (A) | Distearyl dimonium hectorite | 2 | 2 | 2 | 2 | 2 | 2 | 2 | 2 |
| Organic | (Bentone 38VGC) | | | | | | | | |
| modified | | | | | | | | | |
| clay mineral | | | | | | | | | |
| Water-based | Water | 66 | 66 | 66 | 66 | 66 | 66 | 66 | 66 |
| component | | | | | | | | | |

| | | | | | | | | |
|---|---|---|---|---|---|---|---|---|
| Total (% by mass) | 100 | 100 | 100 | 100 | 100 | 100 | 100 | 100 |
| (C) Nonpolar oil/(B) Polar oil | 2.5 | 2.5 | 2.5 | 2.5 | 2.5 | 2.5 | 2.5 | 2.5 |
| Evaluation: Galation stability | C | A | A | A | D | A | A | D |

| | | Ex. | Ex. | Ex. | Comp. | Ex. | Ex. |
|---|---|---|---|---|---|---|---|
| | Component | 7 | 8 | 9 | Ex. 3 | 10 | 11 |
| (C) | Isododecane | — | — | — | — | — | — |
| Nonpolar | Hydrogenated polydecene (silkflo362) | 20 | 20 | 20 | 20 | 6 | 14 |
| oil | Caprylyl methicone | — | — | — | — | — | — |
| | Squalane | — | — | — | — | — | — |
| | Dimethicone (silicone KF-96L-1.5 cs) | — | — | — | — | — | — |
| (B) | Ethylhexyl salicylate (octyl | 8 | 8 | 8 | 8 | 22 | 14 |
| Polar oil | salicylate) (IOB = 0.6) | | | | | | |
| | Octocrylene (IOB = 0.32) | — | — | — | — | — | — |
| | Diisopropyl sebacate (IOB = 0.4) | — | — | — | — | — | — |
| | (Eceran 200) | | | | | | |
| | Isopropyl myristate (IOB = 0.18) (IFM-R) | — | — | — | — | — | — |
| (D1) | PEG-10 hardened castor oil | 2 | 2 | — | — | 2 | 2 |
| POE | (Nikkol HC-10) | | | | | | |
| hardened | PEG-40 hardened castor oil | — | — | 2 | 2 | — | — |
| castor oil | (Nikkol HC-40) | | | | | | |
| (D2) | PEG-10 dimethicone | — | — | — | 2 | — | — |
| Activating | Lauryl PEG-9 polydimethyl | — | — | 2 | — | 2 | 2 |
| agent | siloxyethyl dimethicone (KF-6038) | | | | | | |
| | Polyglyceryl-6 polyricinoleate | 2 | — | — | — | — | — |
| | Cetyl PEG/PPG-10/1 dimethicone | — | 2 | — | — | — | — |
| | (ABIL EM 180) | | | | | | |
| (A) | Distearyl dimonium hectorite | 2 | 2 | 2 | 2 | 2 | 2 |
| Organic | (Bentone 38VGC) | | | | | | |
| modified | | | | | | | |
| clay mineral | | | | | | | |
| Water-based | Water | 66 | 66 | 66 | 66 | 66 | 66 |
| component | | | | | | | |

| | | | | | | |
|---|---|---|---|---|---|---|
| Total (% by mass) | 100 | 100 | 100 | 100 | 100 | 100 |
| (C) Nonpolar oil/(B) Polar oil | 2.5 | 2.5 | 2.5 | 2.5 | 0.27 | 1 |
| Evaluation: Galation stability | A | A | B | D | A | A |

TABLE 2

| | Component | Ex. 12 |
|---|---|---|
| (C) | Isododecane | — |
| Nonpolar oil | Hydrogenated polydecene | 20 |
| | (Silkflo 362) | |
| | Caprylyl methicone | — |
| | Squalane | — |
| | Dimethicone (silicone | — |
| | KF-96L-1.5 cs) | |
| (B) | Ethylhexyl salicylate | 8 |
| Polar oil | (octyl salicylate) | |
| | (IOB = 0.6) | |
| | Octocrylene | — |
| | (IOB = 0.32) | |
| | Diisopropyl sebacate | — |
| | (IOB = 0.4) (Eceran 200) | |
| | Isopropyl myristate | — |
| | (IOB = 0.18) (IPM-R) | |
| (D) | Triisostearic acid | 4 |
| Fatty acid ester | PEG-10 hardened | |
| of POE hardened | castor oil | |
| castor oil | | |
| (A) | Distearyl dimonium | 2 |
| Organic | hectorite | |
| modified | (Bentone38VGC) | |
| clay mineral | | |
| Water-based | Water | 66 |
| component | | |

| | |
|---|---|
| Total (% by mass) | 100 |
| (C) Nonpolar oil/(B) Polar oil | 2.5 |
| Evaluation: Gelation stability | A |

### SPECIFIC PRESCRIPTION EXAMPLES

(Prescription examples 1 to 4) of the water-in-oil type composition according to the present embodiment are presented below.

TABLE 3
(Prescription example 1: Makeup base)

| | Content |
|---|---|
| Component | (% by mass) |
| Hydrogenated didecene | 10 |
| Hydrogenated polydecene | 5 |
| Isododecane | 5 |
| Isohexadecane | 5 |
| Ethylhexyl salicylate | 5 |
| Octocrylene | 5 |
| Distearyldimonium chloride | 0.1 |
| Palmitic acid | 0.05 |
| PEG-10 hydrogensted castor oil | 2 |
| Lauryl PEG-9 polydimethylsiloxyethyl dimethicone | 2 |
| Distearyl dimonium hectorite | 2 |
| Fatty acid-treated titanium oxide | 5 |
| Silicone-treated zinc oxide | 12 |
| Silicone-treated silica | 6 |
| Ester/amino acid-treated pigment-grade titanium oxide | 4 |
| Ester/amino acid-treated yellow iron oxide | 1.2 |
| Ester/amino acid-treated red iron oxide | 0.2 |
| Ester/amino acid-treated black iron oxide | 0.05 |
| Water | 22.5 |
| Hibiscus flower extract | 0.1 |
| Hydrolyzed silk | 0.1 |
| Wild thyme extract | 0.1 |
| 2K glycyrrhizinate | 0.1 |
| Tocopherol acetate | 0.1 |
| Gambir extract | 0.1 |
| Panax ginseng root extract | 0.1 |
| Saxifraga stolonifera extract | 0.1 |
| Bupleurum falcatum root extract | 0.1 |
| 2-O-ethyl ascorbic acid | 0.1 |
| Scutellaria baicalensis root extract | 0.1 |
| Mangosteen bark extract | 0.1 |
| Ethanol | 2 |
| Phenoxyethanol | 0.5 |
| EDTA-3Na | 0.2 |
| Glycerin | 4 |
| Total (% by mass) | 100 |

TABLE 4
(Prescription example 2: BB cream)

| | Content |
|---|---|
| Component | (% by mass) |
| PEG-10 dimethicone | 3.5 |
| Lauryl PEG-9 polydimethylsiloxyethyl dimethicone | 2 |
| PEG-10 hydrogenated castor oil | 2 |
| Ethylhexyl salicylate | 10 |
| Isohexadecane | 10 |
| Hydrogenated didecene | 20 |
| Distearyl dimonium hectorite | 2 |
| Fatty acid-treated titanium oxide | 4.5 |
| Silica | 5 |
| Silicone acid-treated pigment-grade titanium oxide | 4.4 |
| Silicone acid-treated red iron oxide | 0.348 |
| Silicone acid-treated yellow iron oxide | 0.756 |
| silicone acid-treated black iron oxide | 0.0125 |
| Water | 20.2835 |
| EDTA-3Na | 0.2 |
| Xylitol | 1 |
| Trehalose | 1 |
| Glycerin | 2.5 |
| DPG | 7 |
| BG | 2 |
| Ethanol | 1 |
| Phenoxyethanol | 0.5 |
| Total (% by mass) | 100 |

TABLE 5
(Prescription example 3: Sunscreen agent)

| | Content |
|---|---|
| Component | (% by mass) |
| Hydrogenated didecene | 10 |
| Hydrogenated polydecene | 5 |
| Isododecane | 5 |
| Isohexadecane | 5 |
| Ethylhexyl salicylate | 5 |
| Octocrylene | 5 |
| Diisopropyl sebacate | 5 |
| PEG-10 hydrogenated castor oil | 2 |
| Lauryl PEG-9 polydimethylsiloxyethyl dimethicone | 2 |
| Distearyl dimonium hectorite | 2 |
| Hydrophobized titanium oxide | 5 |
| Hydrophobized zinc oxide | 12 |
| Hydrophobized silica | 6 |
| Polyhydroxybutyric acid | 0.1 |
| Poly(3-hydroxybutyric acid-co-3-hydroxyvaleric acid) | 0.1 |
| Water | 24.1 |
| Ethanol | 2 |
| Phenoxyethanol | 0.5 |
| EDTA-3Na | 0.2 |
| glycerin | 4 |
| Total (% by mass) | 100 |

TABLE 6
(Prescription example 4: BB cream)

| | Content |
|---|---|
| Component | (% by mass) |
| PEG-10 dimethicone | 3.5 |
| Lauryl PEG-9 polydimethylsiloxyethyl dimethicone | 2 |
| PEG-10 hydrogenated castor oil | 2 |
| Ethylhexyl salicylate | 10 |
| Isohexadecane | 10 |
| Hydrogenated didecene | 20 |
| Distearyl dimonium hectorite | 2 |
| Fatty acid-treated titanium oxide | 4.5 |
| Silica | 5 |
| Silicone acid-treated pigment-grade titanium oxide | 4.4 |
| Silicone acid-treated red iron oxide | 0.348 |
| Silicone acid-treated yellow iron oxide | 0.756 |
| Silicone acid-treated black iron oxide | 0.0125 |
| Water | 15.2835 |
| EDTA-3Na | 0.2 |
| Niacinamide | 5 |
| Xylitol | 1 |
| Trehalose | 1 |
| Glycerin | 2.5 |
| DPG | 7 |
| BG | 2 |
| Ethanol | 1 |
| Phenoxyethanol | 0.5 |
| Total (% by mass) | 100 |

The present application claims priority to Japanese Patent Application No. 2021-022875 filed Feb. 16, 2021, the entire contents of which are incorporated herein by reference.

1. A water-in-oil type composition, comprising:
(A) an organic modified clay mineral;
(B) 3% by mass or greater of a polar oil having an Inorganic/Organic Balance (IOB) of 0.3 or higher;
(C) a nonpolar oil having an alkyl side chain;
(D1) a polyoxyethylene hardened castor oil; and
(D2) an emulsification activating agent, that is a compound having an alkyl side chain containing 10 or more carbon atoms, or a compound having a fatty acid side chain containing 10 or more carbon atoms or a polyoxyethylene adduct of the compound.

2. The water-in-oil type composition according to claim 1,
wherein (D2) the emulsification activating agent is any selected from the group consisting of:
silicone having an alkyl side chain containing 10 or more and 20 or less carbon atoms; and
a fatty acid ester of glycerin or polyglycerin having a fatty acid side chain containing 10 or more and 24 or less carbon atoms, or a polyoxyethylene adduct of the fatty acid ester, the fatty acid side chain being a group derived from a fatty acid or a group derived from a condensate of an unsaturated fatty acid or a hydroxy fatty acid.

3. The water-in-oil type composition according to claim 1,
wherein a ratio of a content of (D1) the polyoxyethylene hardened castor oil to a content of (A) the organic modified clay mineral is 0.1 or greater and 5 or less.

4. The water-in-oil type composition according to any claim 1,
wherein an average number of moles of ethylene oxide added to (D1) the polyoxyethylene hardened castor oil is from 5 through 60.

5. A water-in-oil type composition, comprising:
(A) an organic modified clay mineral;
(B) 3% by mass or greater of a polar oil having IOB of 0.3 or higher;
(C) a nonpolar oil having an alkyl side chain; and
(D) a fatty acid ester of a polyoxyethylene hardened castor oil.

6. The water-in-oil type composition according to claim 5,
wherein (D) the fatty acid ester of the polyoxyethylene hardened castor oil is a triester of a branched fatty acid containing 13 or more and 25 or less carbon atoms.

7. The water-in-oil type composition according to claim 5,
wherein a ratio of a content of (D) the fatty acid ester of the polyoxyethylene hardened castor oil to a content of (A) the organic modified clay mineral is 0.2 or greater and 10 or less.

8. The water-in-oil type composition according to claim 5,
wherein an average number of moles of ethylene oxide added to (D) the fatty acid ester of the polyoxyethylene hardened castor oil is from 5 through 60.

9. The water-in-oil type composition according to any claim 1,
wherein (B) the polar oil contains an ultraviolet absorbing agent.

10. The water-in-oil type composition according to claim 1,
wherein a number of carbon atoms in the alkyl side chain of (C) the nonpolar oil is 2 or greater.

11. The water-in-oil type composition according to claim 1,
wherein (C) the nonpolar oil is one or more selected from the group consisting of hardened polydecene, caprylyl methicone, and squalane.

12. The water-in-oil type composition according to claim 11,
wherein (C) the nonpolar oil is hardened polydecene.

13. The water-in-oil type composition according to claim 1,
wherein a ratio of a content of (C) the nonpolar oil to a content of (B) the polar oil is 0.25 or greater and 4 or less.

14. The water-in-oil type composition according to claim 1,
wherein a content of a cyclic silicone is 1% by mass or less.

15. The water-in-oil type composition according to claim 1,
wherein the water-in-oil type composition contains 2% by mass or less of a wax, or is free of a wax.
